(19) United States
(12) Patent Application Publication
(10) Pub. No.: US 2003/0135198 A1
(43) Pub. Date: Jul. 17, 2003

(54) Catheter device having multi-lumen reinforced shaft and method of manufacture for same

(75) Inventors: Steven Berhow, St. Michael, MN (US); Brent Anderson, Delano, MN (US); Richard S. Riddle, Rogers, MN (US)

Correspondence Address: EDWARDS & ANGELL, LLP, P.O. BOX 9169, BOSTON, MA, 02209, US

(73) Assignee: TFX Medical Extrusion Products

(21) Appl. No.: 10/319,743

(22) Filed: Dec. 13, 2002

Related U.S. Application Data
(63) Continuation of application No. 09/360,004, filed on Jul. 23, 1999.

(51) Int. Cl. A61M 25/00

(52) U.S. Cl. 604/524; 264/239

(57) ABSTRACT

The present invention provides an improved catheter device having a multi-lumen, reinforced catheter shaft construction. Each lumen is defined by a lubricious liner which promotes the passage of devices or solutions through the lumens with a minimum amount of resistance. A variably flexible outer jacket minimizes trauma to the vascular system of the patient, and offers the attendant medical personnel a high degree of torsional control with respect to the catheter. Methods for the manufacture of such devices are also disclosed.

### BACKGROUND OF THE INVENTION

1. Field of the Invention

The present invention relates to an improved catheter device having a multi-lumen, reinforced catheter shaft construction. Each lumen is defined by a lubricious liner which promotes the passage of devices or solutions through the lumens with a minimum amount of resistance. Methods for the manufacture of such devices are also disclosed.

2. Background

Catheters and other introducer devices are routinely used in a variety of medical and surgical procedures for both diagnostic and therapeutic reasons.

Generally, catheters must be constructed with sufficient flexibility so as to present minimal trauma to the vasculature of the patient. Some degree of stiffness and rigidity also are necessary in order for the catheter to be easily advanced through the vasculature of the patient with a high degree of torsional control.

It is recognized that stiffness and rigidity in the catheter tip pose significant danger to the patient, e.g., puncturing, rupturing or otherwise damaging the vasculature of the patient. Accordingly, some attention has been directed to developing catheters with a soft or relatively flexible distal tip in order to reduce the possibility of such damage.

For instance, U.S. Pat. No. 5,221,270 (Parker) describes a guiding catheter having a soft tip for atraumatic insertion into coronary vessels that is suitable for introduction of an angioplasty balloon catheter.

See also, U.S. Pat. No. 5,234,416 (Macauley) which describes a guiding catheter having a non-traumatic distal tip which is reported as minimizing trauma to the arterial lining; and U.S. Pat. No. 5,792,124 (Horrigan) for its report of a reinforced catheter having a softer distal tip construction.

Catheters with softer distal tip segments, however, present notable disadvantages. For example, a substantially weaker bond may necessarily exist between the soft tip and the less flexible, distal end of the catheter shaft. This is largely due to the thin catheter shaft walls (e.g., walls of less than 0.3 mm in thickness) and to the lower tensile strength of the softer tip materials.

Recognizing that particular disadvantage, certain soft-tip catheters were developed which reported an improved bonding construction. See, for example, U.S. Pat. No. 5,769,830. That patent describes a soft tip guiding catheter which incorporates matching external and internal tapers and cooperating bonding surfaces for increasing the bonding area of the respective surfaces and minimizing the likelihood of separation between the soft tip and tubular portion of the catheter.

Another disadvantage observed in many catheters having a thin-walled, reduced diameter construction is kinking or bending of the catheter. If the catheter becomes kinked or bent, the device must be removed. A new catheter must be inserted into the vasculature of the patient at the same or a different location, and the procedure restarted. This is particularly problematic in emergency situations where time is of the essence, and in the case of patients who must undergo such procedures on a regular basis, as alternate sites for vascular access may be quite limited.

Certain other devices have been developed that are reported to exhibit flexibility and kink-resistance, while presenting minimal trauma to the vasculature of the patient.

For example, U.S. Pat. No. 5,066,285 (Hillstead) describes a catheter introducer sheath made of expanded fibrous polytetrafluoroethylene polymers and similar materials. That patent reports that the use such materials provides a highly flexible, non-kinking sheath.

Another sheath introducer device is described in U.S. Pat. Nos. 5,700,253 and 5,380,403 (Parker). These patents report a flexible, kink-resistant, introducer sheath suitable for percutaneous vascular access and methods for the manufacture of such a sheath. In one embodiment, the introducer sheath includes a flat wire coil which is compression fitted about an inner polytetrafluoroethylene tube.

Despite the many advances in this field and the various devices currently available, there remains a need for an improved catheter device that can facilitate smooth and non-traumatic passage of devices or solutions into the vasculature of a patient with a minimum amount of resistance. Further, it would be highly desirable to develop such an improved device having a construction which is resistant to kinking and bending, and which is variably flexible along the length of the catheter. It would also be highly desirable to develop an improved catheter having a multiple lumen construction, it being possible to vary the shape of the individual lumens to accommodate the introduction of various devices and solutions.

### SUMMARY OF THE INVENTION

The present invention provides an improved catheter device for inserting devices or solutions (or both) into the vasculature of a patient with minimal trauma. Devices of the present invention comprise a kink-resistant, reinforced catheter shaft having a plurality of interior lumens. A variably flexible outer jacket surrounds the reinforced catheter shaft.

Catheters of the present invention are particularly useful when more than one working channel or lumen is required.

Each lumen is defined by a lubricious liner which presents a smooth surface with minimum resistance to the devices or solutions being introduced through the catheter, and which also is resistant to blood clot formation.

In preferred embodiments of the present invention, the lubricious liner comprises a fluoropolymer material. Particularly preferred fluoropolymers include polytetrafluoroethylene and fluorinated ethylenepropylene polymers. Most preferably, the lubricious liner comprises polytetrafluoroethylene.

In particularly preferred embodiments of the present invention, the outer surface of the lubricious liner is etched or otherwise modified to improve the adhesion characteristics of the material.

The reinforcing member reduces the possibility of kinking or bending of the catheter during and after entry into the vasculature of the patient. In preferred embodiments of the present invention, the reinforcing member may comprise round or profiled materials, such as flat stainless steel wire. These materials may be braided in different patterns or densities to provide a custom degree of kink resistance, torque or both. The reinforcement is preferably not compression fit around the underlying catheter; instead, the reinforcement member has a larger inner diameter than the catheter outer diameter.

In alternate preferred embodiments of the present invention, the reinforcing member comprises Nitinol, Kevlar or a polymeric monofilament type of material.

In yet another preferred embodiment of the present invention, the pitch of the braiding or coil may be varied in order to produce a reinforcing member with non-uniform spacing between the braiding or coil turns. Such pitching provides yet another way to vary the flexibility and torquability of the catheter in order to tailor the device to a particular use, procedure or access site, etc.

In preferred embodiments of the present invention, an outer layer, e.g., an extruded polymer jacket, surrounds the outer surface of the catheter. Preferably, the jacket comprises a polymeric material, e.g. a polyurethane, polyethylene, polyester, nylon, nylon copolymer such as a polyetherblockamide (PEBA), and the like.

The outer jacket can also be comprised of numerous segments, each with differing durometers so that the shaft stiffness can be varied from one end of the catheter to the other, for example, to create a desired degree of transition from stiff to flexible. In that way, the present invention provides a catheter which is easy to handle and maneuver, and that is non-traumatic to the vasculature of the patient.

In yet another embodiment of the present invention, the jacket further comprises a radiopaque filler blended into the polymeric material before extrusion.

Methods of manufacturing also are provided to produce an improved catheter with a kink-resistant, reinforced catheter shaft having a plurality of interior lumens which are surrounded by a lubricious liner.

In preferred aspects, such methods generally include the steps of applying, e.g., slipping, the lubricious liners over a profiled supporting mandrel to construct the catheter shaft, applying a reinforcing member over the lubricious liners, applying an outer jacket to the length of the reinforced catheter shaft, applying a covering of heat shrinkable tubing over the assembly, applying heat to the assembly, recovering the shrinkable tubing and removing the supporting mandrels from the inside of each lumen.

In preferred embodiments of the present invention, such methods further comprise altering segments of extruded outer jacket each with differing durometers so that the shaft stiffness can be varied from one end of the catheter shaft to the other, for example.

Other aspects of the invention are disclosed infra.

### BRIEF DESCRIPTION OF THE DRAWINGS

FIG. 1 is a side view of a catheter device of the present invention.

FIG. 2 is a partially cross-sectioned side view of the catheter device of FIG. 1.

FIG. 3 is an alternate, partially cross-sectioned side view of the catheter device of FIG. 1.

FIGS. 4A and 4B shows further preferred reinforced catheter of the invention.

FIG. 5 shows an additional preferred reinforced catheter of the invention.

### DETAILED DESCRIPTION OF THE INVENTION

As discussed above, the present invention provides an improved catheter device having a multi-lumen, reinforced catheter shaft construction. The multi-lumen catheter construction is preferred when more than one working channel or lumen is required for a particular medical or surgical procedure. In that way, only one catheter needs to be inserted into the patient.

Each lumen is defined by a lubricious liner which promotes the passage of devices or solutions (or both) through the lumens with a minimum amount of resistance. Catheter devices of the present invention incorporate a reinforcing member for kink-resistance and a variably-flexible outer jacket. This variable flexibility minimizes trauma to the vascular system of the patient, and offers the attendant medical personnel a high degree of torsional control with respect to the catheter.

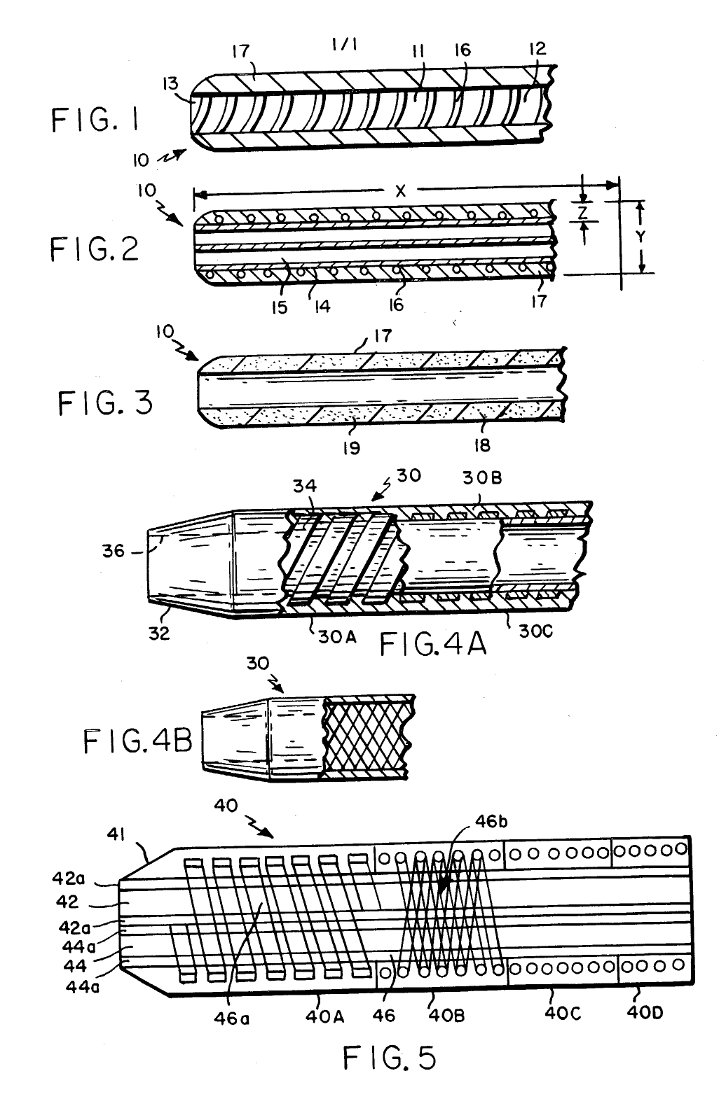

Referring now to FIGS. 1 and 2, a catheter device 10 of the present invention is shown to include a shaft 11 having a proximal end 12 and a distal end 13; a lubricious liner 14 defining a plurality of lumens 15. (In accordance with conventional practice regarding medical devices, “proximal end” designates that end which is closest to the medical personnel manipulating the device, and “distal end” designates the opposite end that is placed within a patient.) The lubricious liner 14 is surrounded by reinforcing member 16, and an outer jacket 17.

The components of the catheter of present invention may be made from a number of materials as will be appreciated by those skilled in the art.

In certain preferred embodiments, catheter 10 has dimensions of about 12 to 48 in length (distance x in FIG. 2) and about 0.053 inches (4 French) to 0.263 (20 French) in diameter (distance y in FIG. 2). Other dimensions, including longer sheaths, also will be suitable.

Preferably, the composite walls of the catheter range from about 0.004 inches to about 0.12 inches, more preferably, from about 0.004 inches to about 0.008 inches in thickness (distance z in FIG. 2).

Catheter shaft 11 is constructed by applying, e.g., slipping, lubricious liners 14 over a profiled supporting mandrel (not shown). The desired number of lumens determines the mandrel profile so that the composite construction represents the desired overall shape of the catheter shaft profile.

Typically, this profile shape is round but it can be oval or some other geometric derivative. For example, if a round catheter shaft profile is desired in a two-lumen configuration, then one of the two mandrels will typically have a crescent shape and one will be round. The round mandrel will be sized to fit into the crescent shape so that the composite profile will be approximately round.

In preferred embodiments of the present invention, the lubricious liner 14 comprises a fluoropolymer material. Particularly preferred fluoropolymers include polytetrafluoroethylene and fluorinated ethylenepropylene polymers. Most preferably, the lubricious liner comprises polytetrafluoroethylene.

In another preferred aspect of the present invention, the outer surface of the lubricious liner 14 is etched or otherwise modified to improve the adhesion characteristics of the material.

Once the lubricious liners 14 have been placed over the supporting mandrels, the mandrels are manually bundled and fed into a braider that will apply a reinforcing member 16 over the lubricious liners 14. The reinforcing member 16 reduces the possibility of kinking or bending of the catheter during and after entry into the vasculature of the patient.

A physical wrapping of the bundles of liners and mandrels also can be utilized. More specifically, a heat shrink coating can be applied over bundles of liners and mandrels prior to feeding same into the assembly into a braider. Suitable materials for forming such a thin-walled heat shrink include e.g. PET or a fluoropolymer such as polytetrafluoroethylene or fluorinatedethylenepropylene.

Preferably, the braider unit includes a facilitating mechanism for entry of the bundle of liners, e.g. a pair of rollers to uniformly fed the bundles into the braider apparatus.

In preferred embodiments, the reinforcing member 16 comprises round or profiled materials, such as flat or rounded stainless steel wire. MP35, a stainless alloy, is another suitable material for construction of the reinforcement member. These materials may be braided in different patterns or densities to provide a custom degree of kink resistance, torque or both.

In alternate preferred embodiments of the present invention, the reinforcing member 16 comprises Nitinol, Kevlar or a polymeric monofilament type of material, such as a nylon, or other polymeric material.

In yet another aspect of the present invention, the reinforcing member 16 may be terminated proximal to the distal end of the catheter shaft, and a spiral reinforcing member (a helical coil of flat or round material) can be manually slid into its place. This embodiment of the present invention is particularly useful when kink resistance and improved flexibility is needed at the distal tip 13 of the catheter 10.

The pitch of the braiding or coil may be varied in order to produce a reinforcing member with non-uniform spacing between the braiding or coil turns. Such pitching provides yet another way to vary the flexibility and torquability of the catheter 10 in order to tailor the device to a particular use, procedure or access site, etc. For example, suitably the spacing between braiding or coil turns of the reinforcements will vary from about 0.010 to 0.050 inches over the length of the reinforcement. The pitch also will preferably vary over defined regions of the reinforcement member. Hence, for example, for a four inch reinforcement, the first inch of the member proximal end may suitably have a 0.010 spacing between coils, the next two inches may have a spacing of 0.020 inches between coils and the final inch may have a spacing of 0.025 inches between coils.

Once the braid or combination of reinforcing members has been applied to the composite, mandrel supported liners, an extruded outer jacket 17 is applied to the entire length of the reinforced shaft 11 by sliding it in place over the reinforcing member 16.

In preferred embodiments of the present invention, outer jacket 17 surrounds the outer surface of the catheter. The outer jacket comprises a polymeric material, e.g. a polyurethane, polyethylene, polyester, nylon, nylon copolymer such as a polyetherblockamide (PEBA), and the like. Such materials of construction can be used in a variety of durometers as desired.

Referring now to FIG. 3, in particularly preferred embodiments of the present invention, the outer jacket 17 comprises numerous segments 18, each with differing durometers so that the shaft stiffness can be varied from one end of the catheter to the other. The segments 18 may be comprised of the same or different material.

The number of differing segments 18 which form outer jacket 17 can range from two to as many as required, but typically includes up to ten. These segments 18 are slid into place over the length of the catheter shaft 11 and are of appropriate length and in the appropriate order to create the desired degree of transition from stiff to flexible. This particular feature enables one to readily alter the flexibility of the catheter. In that way, the present invention provides a catheter which is easy to handle and maneuver, and that is non-traumatic to the vasculature of the patient.

In particularly preferred embodiments of the present invention, the distal end 13 of the catheter 10 is more flexible relative to the shaft portion of the catheter. This construction further provides for non-traumatic entry of the device into the vasculature of the patient.

In yet another embodiment of the present invention, the outer jacket 17 further comprises a radiopaque filler 19. Typically, the radiopaque filler 19 is blended into the polymeric material of the jacket prior to extrusion. Preferably, this filler ranges in percentages from about 5% to about 40% by weight and comprises barium sulfate, tungsten, bismuth sub-carbonate or bismuth trioxide. Such a configuration permits visualization of the catheter within a patient by x-ray or fluoroscopic procedures.

Catheter 10 also may comprises a radiopaque tracer ring (not shown), preferably positioned at or proximate to the distal tip of the sheath. Use of such a radiopaque marker permits visualization of the sheath distal end within a patient by x-ray or fluoroscopic procedures.

FIG. 4A shows a preferred catheter 30 of the invention having a segmented portions of different hardness. The catheter 30 includes tapered distal tip 32 and exterior reinforcement member 34 that preferably terminates before tip 32 as depicted in FIG. 4A. As discussed above, the reinforcement member suitably may have a variety of configurations, such as a generally flat wire spiral as shown in FIG. 4A, or a round wire braid as shown in FIG. 6B. Also, other wrapping configurations will be suitable with those materials, e.g., a round wire can be configured as a spiral reinforcement, and the flat wire can be configured as a braided reinforcement.

Catheter 30 also has segments of varying hardness, specifically distal segment 30A is comparatively the least hard portion of the sheath; a middle sheath segment 30B that has an intermediate hardness and greater hardness than distal segment 30A; and a proximal segment 30C that is the most hard the three depicted segments. The catheter also has lubricous inner liners 36 such as PTFE or other fluoropolymer for the entire catheter length.

FIG. 5 shows a further preferred catheter 40 of the invention that has tapered distal end 41 and includes multiple lumens 42 and 44 that include lubricous liners 42a and 44a respectively, preferably a fluorinated materials as discussed above. Catheter 40 includes reinforcement member 46 that includes a coiled portion 46a and braided section 46b. Each of coiled portion 46a and braided section 46b may be flat wire or round wire, or other configured wrapped reinforcing material.

Catheter 40 also preferably includes segments along the catheter that differ in hardness. More particularly, distal catheter segment 40A is typically constructed to be the softest portion of the several longitudinal catheter segments; segment 40B is suitably harder and/or constructed of different material(s) than distal segment 40A; segment 40C is suitably harder and/or constructed of different material(s) than distal segment 40B; and proximal segment 40D is suitably harder and/or constructed of different material(s) than distal segment 40C.

As discussed above, the invention also provides methods of manufacturing an improved catheter with a kink-resistant, reinforced catheter shaft having a plurality of interior lumens which are surrounded by a lubricious liner.

In preferred aspects, such methods generally include the steps of applying, e.g., slipping, the lubricious liners over a profiled supporting mandrel to construct the catheter shaft, applying a reinforcing member over the lubricious liners, applying an outer jacket to the length of the reinforced catheter shaft, and molding the jacket to the reinforced catheter shaft.

In preferred embodiments of the present invention, such methods further comprise altering segments of the outer jacket with material(s) having differing durometers or materials so that the shaft stiffness can be varied from one end of the catheter shaft to the other.

Once the outer jacket or jacket segments are in place, a covering of heat shrinkable tubing is applied over the entire assembly. Preferably, the heat shrinkable tubing comprises at least one of a fluorinated ethylene propylene or polytetrafluoroethylene polymer.

The heat shrinkable tubing is recovered by applying heat from an external source, procedures for which are well known to those skilled in the art. This assembly is passed through a heated die of a controlled size and at a controlled rate to heat fuse the outer jacket segments with each other. The outer jacket is also melted through the reinforcing member and bonded to the etched outer surface of the lubricious liner.

The final stage involves removing the heat shrinkable tubing from the outside of the assembly and removing the supporting mandrels from the inside of each lumen.

Another application would be in the construction of a catheter with a steerable distal tip. These devices typically use wires attached to the catheter handle and distal tip to move the tip at an angle from the centerline. When these wires are articulated back and forth, the tip of the catheter is deflected and directed to an appropriate anatomical location. Additionally, the multi-lumen construction of catheter of the invention provides for use of one, two or more smaller lumens as passageways for wires to articulate the distal tip.

The novel design of the present invention provides an improved catheter device that incorporates a multi-lumen, reinforced catheter shaft construction. A reinforcing member is also included for kink-resistance. A variably flexible outer jacket minimizes trauma to the vascular system of the patient, and offers the attendant medical personnel a high degree of torsional control with respect to the catheter.

The terms and expressions which have been employed herein are used as terms of description and not of limitation. There is no intent, in the use of such terms and expressions, of excluding any of the equivalents of the features shown and described or portions thereof, but it is recognized that various modifications are possible within the scope of the invention claimed.

What is claimed is:

1. A catheter comprising:
(a) a catheter shaft having a proximal end and a distal end;
(b) a plurality of lumens contained within the catheter shaft;
(c) a lubricious liner surrounding each of the lumens;
(d) a reinforcing member surrounding the lubricious liners;
(e) an outer jacket surrounding the reinforcing member and extending longitudinally along the length of the catheter shaft.

2. The catheter of claim 1, wherein the lubricious liner comprises a fluoropolymer.

3. The catheter of claim 2, wherein the fluoropolymer comprises a polytetrafluoroethylene polymer or a fluorinated ethylene-propylene polymer.

4. The catheter of claim 1, wherein the lubricious liner is etched or otherwise modified on an outer surface thereof.

5. The catheter of claim 1, wherein the reinforcing member comprises at least one of a round or profiled stainless steel material.

6. The catheter of claim 1, wherein the reinforcing member comprises at least one of a Nitinol, Kevlar or polymeric monofilament type material.

7. The catheter of claim 1, wherein the reinforcing member terminates proximal to the distal end of the catheter shaft.

8. The catheter of claim 1, wherein the outer jacket comprises a polymeric material.

9. The catheter of claim 8, wherein the polymeric material comprises at least one of a polyurethane, polyethylene, polyester, nylon, or nylon copolymer.

10. The catheter of claim 1, wherein the outer jacket comprises a plurality of segments having differing durometers.

11. The catheter of claim 10, wherein the plurality of segments ranges from about two to about ten segments.

12. The catheter of claim 8, wherein the outer jacket further comprises a radiopaque filler material.

13. The catheter of claim 12, wherein the radiopaque filler material comprises at least one of barium sulfate, tungsten, bismuth sub-carbonate or bismuth trioxide.

14. The catheter of claim 12, wherein the radiopaque filler material ranges in percentages from about 5% to about 40% by weight.

15. The catheter of claim 10, wherein the distal end of the catheter is more flexible relative to the shaft portion of the catheter.

16. A method of manufacturing a catheter comprising the steps of:
(a) applying lubricious liners to a profiled supporting mandrel to form a catheter shaft;
(b) applying a reinforcing member over the lubricious liners;
(c) applying an outer jacket along the length of the reinforced catheter shaft; and
(d) molding the outer jacket to the reinforced catheter shaft.

17. The method of claim 16, further comprising etching an outer surface of the lubricious liners.

18. The method of claim 16, wherein a wrapping is applied over the liners in step (a).

19. The method of claim 16, wherein a heat shrink wrapping is applied over the liners.

20. The method of claim 19, wherein the wrapping is a fluoropolymer.

21. The method of claim 16, wherein the step of applying the outer jacket further comprises alternating segments of materials having different hardnesses across the length of the catheter shaft.

22. The method of claim 16, wherein the step of molding the jacket to the reinforced catheter shaft comprises substantially covering the outer jacket with heat shrinkable tubing, applying heat from an external source and removing the heat shrinkable tubing from the outside of the outer jacket.

23. The method of claim 16, wherein the step of applying the reinforcing member further comprises altering the pitch or spacing of the reinforcing member.

24. The method of claim 16, wherein the step of applying the reinforcing member further comprises terminating the reinforcing member proximal to the distal end of the catheter.

25. The method of claim 16, further comprising blending a radiopaque filler material into the outer jacket prior to applying the outer jacket to the reinforced catheter shaft.

26. The method of claim 16, further comprising removing the supporting mandrels from the inside of each lumen.
